(19) United States
(12) Patent Application Publication
(10) Pub. No.: US 2006/0280810 A1
(43) Pub. Date: Dec. 14, 2006

(54) Disinfecting teat care compositions

(76) Inventors: Robert D. Kross, Bellmore, NY (US); Lorrence H. Green, Westbury, NY (US)

Correspondence Address: COLEMAN SUDOL SAPONE, P.C., 714 COLORADO AVENUE, BRIDGE PORT, CT, 06605-1601, US

(21) Appl. No.: 10/575,326

(22) PCT Filed: Oct. 15, 2004
(86) PCT No.: PCT/US04/34142

§ 371 Date: Jun. 12, 2006

Related U.S. Application Data
(63) Continuation-in-part of application No. 10/780,435, filed on Feb. 17, 2004.
(60) Provisional application No. 60/511,916, filed on Oct. 17, 2003.

(51) Int. Cl. A61K 33/00; A61K 31/19

(52) U.S. Cl. 424/718; 514/557

(57) ABSTRACT

The present invention relates to novel compositions which are used to produce nitrous acid, in preferred aspects such compositions comprise a protic acid and a metal nitrite, and to methods for using these compositions, in particular for disinfecting mammalian teat skin.

### RELATED APPLICATIONS

This application claims priority from U.S. application Ser. No. 10/780,435, filed Feb. 17, 2004 and provisional application U.S. 60/511,916, filed Oct. 17, 2003, which are incorporated by reference in their entirety herein.

### FIELD OF THE INVENTION

The present invention relates to novel compositions which are used to produce nitrous acid and to methods for using these composition, in particular for disinfecting mammalian teat skin.

### BACKGROUND OF THE INVENTION

One of the single, most powerful controls that a dairy farmer can avail himself of, is routine pre- and post-milking teat dipping. This procedure can dramatically reduce the incidence of mastitis in his dairy herd. Mastitis is by far the most prevalent and costly disease affecting dairy herds. More than half of the dairy animal population is thought to be affected by bovine mastitis to some degree. Mastitis causes a lowering of milk output and a reduced milk quality, accounting for losses in the U.S. alone approaching $2 billion, a major portion of which results from the lowered milk output of infected cows.

Mastitis is literally an inflammation of the mammary gland principally caused by invasion of bacteria through the teat orifice at or around milking times. During the milking process, it results from transfer to the teat, from cow-to-cow or cow-to-human-to-cow, of so-called Contagious microorganisms from contaminated equipment and hands; or at other times, when the teat orifice remains open post-milking, as a result of contact with so-called Environmental microorganisms that deposit on the teat and udder between milking periods. The Contagious organisms are primarily two, Staphylococcus aureus and Streptococcus agalactiae, while Environmental organisms include Streptococcus uberis, Streptococcus dysgalactiae, Klebsiella pneumoniae and Escherichia coli. The latter are present in the cow's surroundings, including the soil, its bedding, feces, and contaminated water.

Treatment of mastitis is more costly, and often ineffective, and generally involves antibiotic therapy. During such periods and for a number of days thereafter, the cow's milk must be discarded. Dairymen uniformly agree that washing and/or disinfecting teats before milking, and dipping the teats afterwards, can markedly reduce the transfer of environmental organisms to the teat, by self-infection, or by post-milking with teat dips to control the transfer of contagious organisms from the equipment and hands. The teat dips (a term used herein to include teat sprays as well) generally embody antimicrobial agents which are capable of at least substantially reducing in number, all such pathogens, and these dips additionally contain such optional agents as humectants, thickening and/or barrier-forming agents, and colorants. Without such disinfection, the individual teat quarters are subject to a greater probability of becoming mastitic, causing problems ranging from lower milk quality and poorer milk yields to the actual death of the afflicted animal.

Teat dips, which are almost invariably liquid compositions, are most often single solutions or suspensions, used directly by the dairyman by withdrawal of a day's portion from a larger container. Two-part teat dips have also been employed since 1987, where the active antimicrobial agent is formed by combination of certain chemical compounds in both parts shortly before application. The antimicrobial agents most often found in single part systems include iodophors, quaternary ammonium compounds, organic sulfonates, and chlorhexidine. The dual-part teat dips generally contain a chlorous acid system

Chlorous Acid Teat Dips: These two part teat dips are based on the generation of chlorous acid and/or chlorine dioxide by combination of a metal chlorite in one part and an acid source in the other, to form chlorous acid. The subsequent degradation of this acid into a series of transient, cidal oxidants provides a heretofore unparalleled means for killing or inactivating a broad spectrum of bacteria, yeasts, molds and viruses in a very rapid manner, and in high numbers. It is generally accepted that the acidified chlorite system is the most up-to-date and effective antimicrobial in teat dip compositions.

More specifically, with regard to the metastable chlorous acid system, the chlorous acid molecule, HClO₂, represents a relatively small fraction of the total chlorite ion present, typically no more than about 15%. This tends to minimize the otherwise rapid degradation of the system. The antimicrobially-effective chlorous acid systems function at pH values from about 3.5 down to about 2.6. The protic acid source to effect this conversion is generally an organic acid (U.S. Pat. Nos. 4,986,990, 5,185,161), although inorganic acids (U.S. Pat. No. RE 36,064) and even acid-inducing metal salts have been taught (U.S. Pat. No. 5,820,822), in the extended series of patents which disclose the various aspects of this technology. The acidified chlorite compositions were first taught by Alliger in 1978 (U.S. Pat. No. 4,084,747) and in 1982 (U.S. Pat. No. 4,330,531), where the acid activator was lactic acid, which was deemed critical to the unique activity of the acid/chlorite system. Subsequent prior art taught the creation of a diverse range of acidified chlorite compositions and their method of use. These included U.S. Pat. No. 4,891,216 (for topical application); U.S. Pat. No. 4,956,184 (for genital herpes); U.S. Pat. No. 5,100,652 (for oral hygiene); U.S. Pat. No. 5,384,134 (for anti-inflammatory activity); U.S. Pat. Nos. 5,389,390 and 6,063,425 (for disinfecting poultry and other meats); U.S. Pat. Nos. 5,597,561 and 5,651,977(adherent topical disinfectants); U.S. Pat. No. 5,628,959 (sterilizing hemodialyzers); U.S. Pat. No. 5,772,985 (bovine warts); U.S. Pat. No. 6,096,350 (for honey bee diseases); and U.S. Pat. No. 6,123,966 (stabilized disinfecting compositions).

Chlorous acid: Positives and Negatives: One of the present inventors, Robert Kross, has worked extensively for a number of years in this area of technology (he is a named inventor on the above-described patents), and as such, he has become very familiar with the capabilities and deficiencies of the acid/oxy anion system, based upon chlorite. Although the capabilities of the acidified chlorite system are extensive, several inherent characteristics are present which limit its application in certain situations. The major difficulty lies in the relatively strong oxidizing tendency of the system, and in particular the corrosive effects of the chlorine dioxide (ClO₂), which forms upon degradation of the chlorous acid. ClO₂ will corrode many of the metals used in dairy spray equipment as well as those used in fabrication of medical and dental equipment, and those used to dispense the food disinfecting solutions. A further detriment of the acidified chlorite systems is the noxiousness of the ClO₂ gas, for which OSHA has listed a very low permissible concentration in the air to which workers may be exposed for an 8 hour period. That level, 0.1 parts per million in the air, is 10 times lower, for example, than for chlorine, for which OSHA has listed a maximum permissible level of 1.0 ppm over an 8-hour period.

Alternative Two-Part Oxyanion System: In researching the seeming uniqueness of the uninegative chlorite ion, it became evident that there is another oxyanion, namely the nitrite ion, that is similar to that of chlorite (see, for example, Friedman's “On the Ultraviolet Absorption Spectra of Uninegative Ions,” re: the electronic properties of both ions). Both form unstable acid counterparts, i.e. chlorous and nitrous acids, with increasing instability as the acid form represents a growing fraction of the acidified oxyanion solution. Neither acid can be isolated. Nitrogen appears in at least 8 oxidation states in its water soluble species; chlorine has at least 6. In general, species such as nitrous and chlorous acids, which have intermediate oxidation numbers, will be unstable with respect to disproportionation. The degradation of both of these acids leads to the formation of gases (chlorine dioxide and nitric oxide [NO]) which are unique in possessing unpaired electrons. Both of these materials have unusual properties.

Chlorine dioxide has become an excellent replacement for chlorine in water disinfection, by virtue of its high biocidal activity without formation of chloro-organic mutagens. It has also found use in the disinfection of food. In both these cases the chlorine dioxide degrades through several steps, through a 5-electron transfer, to innocuous chloride ion. With respect to nitric oxide, while it is one of the simplest biological molecules in nature, it has recently found its way into nearly every phase of biology and medicine. This ranges from its role as a critical endogenous regulator of blood flow and thrombosis, to a principal neurotransmitter mediating erectile function, to a major pathophysiological mediator of inflammation and host defense. These major discoveries have stimulated intense and extensive research into a vast array of fields including chemistry, molecular biology, and gene therapy.

One difference between the two paramagnetic, unpaired-electron molecules of chlorine dioxide and nitric oxide, which derive respectively from the degradation of chlorous acid and nitrous acid solutions, is that the chlorine in chlorine dioxide has lost one electron with respect to that in chlorous acid (i.e. a +4 charge in the former vs. +3 in the latter), whereas the nitrogen in nitric oxide, with a +2 charge, has gained an electron as cf. the +3 charge of the nitrite nitrogen. This difference may lead to an advantage in the potential metal corrosivity of nitrous acid/nitric oxide systems versus chlorous acid/chlorine dioxide systems. In particular, a nitrous acid-based teat spray may well be less corrosive on metal spray equipment than a chlorous acid teat spray.

It appeared appropriate therefore, by virtue of the similar chemistry of the acidified nitrite as cf. the chlorite system, and its possible role in producing antimicrobial activity paralleling that of acidified chlorite, to investigate, determine, and optimize the acidified nitrite system as a disinfecting agent for use in teat dips. The specific intent would be to reduce or eliminate those microorganisms which may otherwise contaminate the teats of cows and other milk-producing species, from milking equipment, hands, and the environment, that could otherwise lead to infection of the mammary gland (i.e. mastitis).

Although nitrous acid compositions for the treatment of infection have been proposed in the art, their applicability for use as teat dip compositions has not been proposed. The prior art composition require that the two components are admixed at the intended environment of use (i.e. the diseased tissue) to release the NO and NO₂ which are the purported active agents. If nitrous acid compositions are to be used for teat dips, on the other hand, it would be necessary to premix the components up to one day prior to use, and have the resulting mixture continue to function antimicrobially. Stability of such solutions would optimally allow for their use for at least a week, so that dairy farmers would not have to continually discard excess teat dip mixtures that have not been applied to the animals, and thus prepare fresh mixtures at least once per day. If the farmer could “top off” remaining mixtures with fresh components, it would save considerably in both time and cost.

To that end, to determine the functionality and applicability of acidified nitrite systems for teat dip or spray use, the following program was instituted. Germicidal activity was evaluated as a function of the nitrous acid/nitrite ratios in test solutions, as controlled by available hydrogen ion. Success in this endeavor would be achieved if:

a)—suitable acid:nitrite combinations were found that were appropriately germicidal against the contagious and environmental organisms associated with mastitis;

b)—both nitrite and acid phases would accommodate, and be sufficiently compatible with, other agents generally used in teat dip formulations; and

c)—most importantly, the resulting acidified nitrite systems had the longevity of action following mixture that it could be used for at least one day thereafter, while maintaining sufficient high germicidal activity that it would be economically viable.

Accordingly, the present invention resulted from a search for a controllable acidified nitrite antimicrobial system to parallel or even exceed the superior qualities of the acidified chlorite system, as has been manifest in the number of successful teat dips based thereon. If, indeed, some of the negative qualities of acidified chlorite teat dips could be improved upon, such as the tendency to lose color intensity, generate noxious odors, corrode metal parts, and lose significant activity within a few hours, the search would be deemed that much more successful.

### OBJECTS OF THE INVENTION

It is, therefore, an object of the present invention to provide antimicrobial acidified nitrite solutions for use as disinfectants in external teat care products.

It is a further object of the invention to control the antimicrobial action of these solutions by modifying the nitrite concentration and acidity, and thus the degree of conversion of nitrite ion to nitrous acid, the presumptive source of germicidal action.

It is a further object of the invention to increase the length of time over which the antimicrobial action of these solutions is exerted, by modifying the nitrite concentration and acidity of these solutions.

It is yet a further object of the invention to provide compositions based upon nitrous acid which exhibit rapid bacterial kill and a broad spectrum of action against representative species of the various microbial types which are of particularly concern in udder health.

It is still another object of the invention to provide acidified nitrite solutions which exhibit significant antimicrobial activity and to reduce and/or completely avoid corrosion of equipment associated with the milking process, including spray devices, milking claws, storage tanks, and pipe lines in comparison to chlorous acid systems.

It is an additional object of the invention to provide acidified nitrite solutions which are well tolerated by animal tissues, particularly those of teat skin.

These, and/or other objects of the present invention will become apparent from a review of the following summary of the invention and description of the preferred embodiments.

### SUMMARY OF THE INVENTION

In one aspect, the present invention provides a composition for disinfecting animal teats and milking-associated equipment, using a nitrous acid generating composition. This composition comprises an aqueous solution containing a suitable amount of a protic acid and a suitable amount of a metal nitrite. The nitrite ion concentration (mole percent) in the form of nitrous acid is no more than about 95% by weight of the total amount of nitrite ion concentration. In certain preferred aspects of the present invention, the concentration (mole percent) of nitrous acid is no less than about 10% and no greater than about 67% (about two-thirds) of the total amount of nitrite ion concentration.

In a preferred embodiment of this aspect of the present invention, there is provided a composition for disinfecting the teats of the mammary glands and milking-associated equipment with a composition comprising a nitrous acid generating compound with a sufficient amount of a suitable organic acid to lower the pH of the composition to less than about 4.5. The preferred organic acid is an α-hydroxy acid having a pKa ranging from about 2.8 to about 4.8 which preferably has the formula:

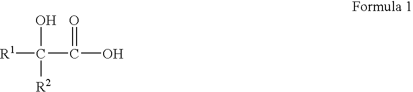

Where R¹ and R² may be the same or different and may be selected from the group consisting of hydrogen, methyl, —CH₂COOH, —CH₂COO—, —CH₂OH, —CHOHCOOH, —C₆H₅, and —CH₂C₆H₅.

In another preferred embodiment of this aspect of the present invention, there is provided a composition for disinfecting the teats of mammary glands and milking-associated equipment with a composition comprising a nitrous acid generating compound with an amount of phosphoric acid (pKₐ=2.15) sufficient to lower the pH of the composition to less than about 4.5.

In another preferred embodiment of this aspect of the present invention, there is provided a storage stable nitrous acid composition for disinfecting the teats of mammary glands and milking-associated equipment with a composition comprising a nitrous acid generating compound, where the composition maintains adequate germicidal activity for a period of at least one week and preferably at least about three weeks after its preparation.

In another aspect, the present invention provides processes for disinfecting the teats of mammary glands and milking-associated equipment using the compositions described above. These processes comprise applying the compositions described above to the teats of mammary glands and milking-associated equipment by dipping, spraying, or immersion as appropriate, in order to disinfect the substrate.

In yet another aspect, the present invention provides a process for preparing these disinfecting compositions and separately, for disinfecting the teats of mammary glands and milking-associated equipment using the resulting nitrous acid containing composition. The process comprises contacting the protic acid with the metal nitrite to form the disinfecting compositions, which are used in effective amounts to disinfect the desired surface.

### DETAILED DESCRIPTION OF THE INVENTION AND PREFERRED EMBODIMENTS

In describing the present invention, the following terms will be used.

The term “nitrite” or “nitrite salt” is used throughout the specification to describe a salt of nitrous acid which is readily soluble in an aqueous system and which readily dissociates into nitrite anion and counterion (generally, metal). Two particularly preferred salts of nitrites for use in the present invention include sodium nitrite and potassium nitrite, although a number of other nitrite salts may also be used in the present invention. The term “nitrite” is used throughout the specification to describe the form in which an amount of a water soluble salt of nitrous acid either in dry or liquid state (preferably, as an aqueous solution) is added to the acid. In general, the nitrite is added to the acid and preferably, both the nitrite and the acid in an aqueous solution are mixed together to which has been added effective amounts of additives such as surfactants, coloring agents, chelating agents and gelling agents, as otherwise described herein. Metal nitrite salts are preferred for use in the present invention.

The term “nitrite ion” is used throughout the specification to describe the nitrite anion of a nitrite salt. In the present application, where the term “nitrite ion” is described in amounts in a given aqueous composition, it is the amount or concentration of the anion which is being referenced, not the amount of total salt concentration which generally contains both a nitrite anion and a metal cation.

The term “acid” is used throughout the specification to describe protic acids, i.e., acids that release hydrogen ions in solution. Acids for use in the present invention include strong inorganic acids such as hydrochloric, sulfuric, and nitric acid; alkylsulfonic acid and benzenesulfonic acid, among other organic sulfonic acids, which, depending upon the end-use of the composition, may be preferably included as dilute acid; organic acids such as citric, fumaric, glycolic, lactic, malic, maleic, tartaric acid, salicylic, citric, propionic, acetic and mandelic, among others, including ethylenediaminetetraacetic acid (EDTA, as the free acid or the monosodium salt), among others; and inorganic acids such as sodium and potassium bisulfate (NaHS0₄ and KHS0₄) and phosphoric acid, among numerous others. It is noted that numerous additional acids may also be used in the present invention. In its broadest aspect, compositions according to the present invention may make use of virtually any acid, to the extent that it provides an initial pH, which when the nitrite-containing part and the acid-containing part are combined produce nitrous acid in amounts effective for the intended purpose. One of ordinary skill will be able to readily determine the type and amount of acid to be used for a particular application.

The term “effective amount” is used to describe that amount of a composition, an individual component or a material which is included in compositions according to the present invention in order to produce an intended effect. For example, in the case of an effective amount of an acid, an effective amount is that amount which is included to produce a sufficiently acidic medium to produce nitrous acid in combination with a nitrite salt An effective amount of nitrite or a nitrite salt is that amount which is effective to produce a desired concentration of nitrous acid after mixing with an appropriate and effective amount of an acid. In the case of a gelling agent, an effective amount of that component is that amount which is effective to gel a final composition (i.e., produce a viscous composition). One of ordinary skill will be able to readily determine effective amounts of components or compositions for use to provide an intended effect.

The term “gelling agent” is used throughout the specification to describe a compound or composition which is added to the present compositions in order to increase the viscosity of the composition. Gelling agents which are used in the present invention may be added to the nitrite-containing part or the acid-containing part in amounts effective to gel the solution to which these compounds have been added. Gelling agents for use in the present invention include polysaccharides produced by microbial cultures such as xanthan, or extracted from legume seeds, such as the galactomannans, including guar gum and locust bean (carob) gum. Other gelling agents include high molecular weight polyoxyalkylene crosslinked acrylic polymers as well as the highly preferred cellulosics such as hydroxymethyl cellulose, hydroxypropyl cellulose, hydroxyethyl cellulose, methyl cellulose, methylpropyl cellulose, among others, including high molecular weight polyethylene glycols, polyacrylainide and polyacrylamide sulfonates, and crosslinked polyvinylpyrrolidones, among others. A gelling agent is used in an effective amount in compositions to increase the viscosity of the composition. In a teat dip aspect of the invention, the amount of gelling agent which is used is that amount which allows the composition to cling to the teat of the cow or other mammal without significant loss of material from the exposed tissue. In preferred aspects of the present invention, the thickened composition remains on the teat upon drying of the composition thereon.

The term “storage stable” or “long-acting” refers to compositions according to the present invention which exhibit significant antimicrobial activity (i.e., the composition can produce at least a 50% microbial kill within a period of no more than about 5 minutes and preferably no more than about a minute or two) for a period of at least about 24 hours, preferably at least about 48 hours, preferably at least about 3 days (72 hours), preferably at least about 1 week (168 hours), and more preferably at least about three weeks (about 500 hours), at least about two months, at least about three months or more. It is at least one important aspect of the present invention to provide storage stable antimicrobial compositions which can be used as storage-stable or long-acting compositions, in order to promote efficiencies and favorable economics of use.

The term “noncorrosive” or “substantially non-corrosive” are used interchangeably within the context of the present invention to describe the favorable characteristic of the invention as reducing or substantially avoiding corrosion of metal containers, which typically occurs with the use of chlorous acid solutions on metal containers and other acid-sensitive equipment, especially milk containers and other dairy equipment.

As described above, the present invention is directed to methods and processes for use of nitrous acid generating compositions to disinfect aninial teats and milking-associated equipment as well as for general disinfection purposes.

The composition comprises an aqueous solution containing a suitable amount of hydrogen ions derived from a protic acid and a suitable amount of a metal nitrite such as sodium nitrite. Compositions according to the present invention are preferably produced by adding a metal nitrite (either as a dry material or in solution) to an acidic aqueous solution. The concentration of hydrogen ion-generating species is such that the amount of nitrite ion in the form of nitrous acid is no more than about 95% by weight of the total nitrite ion in the solution. Preferably, the amount of nitrite in the form of nitrous acid is no more than about 67% by weight of the total nitrite ion concentration in solution.

The percent by weight of nitrite and nitrous acid may be calculated from the ionization constant of nitrous acid and the amount of hydrogen ion in solution produced by partial ionization of the protic acid, or calculated from the pH of a salt-induced acid solution. The hydrogen ion concentration, [H⁺], in a solution of a protic acid, HA, of known molar concentration and whose ionization constant is Ka, may be calculated from the following relationship:

$$Ka = \frac{[ {H}^{+} ] [ {A}^{-} ]}{[ HA ]}$$

The above relationship may be applied to calculate the relative nitrite and nitrous acid concentrations, where the ionization constant for nitrous acid is 4.5×10⁻⁴. That is:

$$4.5 \times {10}^{- 4} = \frac{[ {H}^{+} ] [ {{NO}_{2}}^{-} ]}{[ {HNO}_{2} ]}$$

where the hydrogen ion concentration, [H⁺], is the quantity readily determined by ionization of the known amount of the protic acid, HA. This calculation is well known to those skilled in this art.

For the nitrous acid/nitrite system, the following table illustrates representative percentages of both species over a pH range which provides high to low amounts of nitrous acid, as derived from nitrite.

TABLE 1
Percentage of Nitrite as Nitrous Acid
at Varying pH Values

| pH | Nitrous Acid % | Nitrite % |
|---|---|---|
| 1.5 | 98.4 | 1.6 |
| 2.0 | 95.2 | 4.8 |
| 2.3 | 90.9 | 9.1 |
| 2.6 | 83.3 | 16.7 |
| 2.8 | 76.0 | 24.0 |
| 3.0 | 66.7 | 33.3 |
| 3.3 | 50.0 | 50.0 |
| 3.5 | 38.8 | 61.2 |
| 4.0 | 16.6 | 83.4 |
| 4.5 | 6.0 | 94.0 |
| 5.0 | 2.0 | 98.0 |

Aqueous solutions of nitrous acid are unstable, and decompose according to the following equation. Instability increases with increased absolute and relative molar concentrations of the HONO, and with increasing heat:
3HNO₂(aq)< - - - >H⁺(aq)+2NO(g)+NO₃⁻(aq)+H₂O(l) Equation 1

The reaction is a combination of the two half-reactions, as follows:

$$\begin{matrix}2 \{ {HNO}_{2} + {H}^{+} + {e}^{-} < … > NO + {H}_{2} O \} & Equation 2 \\ \frac{{HNO}_{2} + {H}_{2} O < … > {NO}_{{3}^{-}} + 3 {H}^{+} + 2 {e}^{-}}{\begin{matrix}Σ = 3 {HNO}_{2} ( aq ) < … > {H}^{+} ( aq ) + \\ 2 NO ( g ) + {{NO}_{3}}^{-} ( aq ) + {H}_{2} O ( 1 )\end{matrix}} & Equation 3\end{matrix}$$

In addition to the concentration-dependent degradation of nitrous acid as shown above, nitrous acid will also act as an oxidizing agent in the presence of oxidizable materials, such as microorganisms, according to the first half-cell reaction above (Equation 2), with a redox potential εₒ=1.00 volts. Accordingly nitrous acid systems are quite destructive of all classes of microorganisms which are susceptible to oxidation, including bacteria, yeasts, molds and viruses. This destruction is well known for other non-specific oxidizing germicides such as bleach (hypochlorous acid), chlorous acid, chlorine dioxide, and iodine. We have discovered that acidified nitrite solutions, upon standing, will generally become either more acidic or less acidic in rough proportion to the pH of the solution, and thus the relative amount of nitrous acid with respect to nitrite ion. At relatively high concentrations of nitrous acid with respect to total nitrite (>˜75%), stored solutions will generally become more acidic. At relatively lower concentrations of nitrous acid, the stored solutions will generally become more alkaline (i.e. less acidic). In one set of experiments the break-point with respect to greater or lesser acid formation occurred at ˜pH 3.7, where the total molar concentration of nitrite [ionic and acid-form] was 0.045M/liter. The data were as follows:

| pH at T = 0 | pH at T = 30 days |
|---|---|
| 2.94 | 2.30 |
| 3.12 | 2.50 |
| 3.35 | 3.25 |
| 3.54 | 3.15 |
| 3.75 | 3.92 |
| 3.90 | 4.35 |

The first solution, at pH 2.94, with a relative nitrous acid level of about 70% (see Table 1), increased in acidity to 2.30, a pH drop of 0.64 units, whereas the last solution, at pH 3.90, and a relative nitrous acid level of about 20%, increased in pH by 0.45 units. The quantity of acid required to reduce the pH in the first solution is, of course, much greater than for the last solution, in large measure because of the logarithmic basis for the pH scale.

Although the direct reason for this difference is not fully understood, the increase or decrease of solution pH is believed to be related to the corresponding contributions of half-ell Equations 2) and 3) above, the former reducing the H⁺ present in solution (i.e. raising the pH) and the latter contributing H⁺ to the medium, and thereby lowering the pH. There may be some involvement of the organic acidifier, which in this experiment was malic acid, in the overall reaction characteristic of the particular combination of nitrite and acid concentrations in this set of solutions. However it is evident from this experiment that it is feasible to adjust the concentrations of nitrite and acid in a preferred composition of this invention such that the solution is stabilized in pH over a prolonged period of time, and capable of being stored as a pre-mixed one-part composition. This is a most-important finding, with respect to the use of acidified nitrite solutions for teat care products, as pointed out earlier. This would be of significant benefit to dairy farmers, since a teat dip or spray with this type of stability would obviate the need for daily, or even twice-daily mixing of ample, but not excess quantities sufficient for the particular set of animals that require pre- &/or post-milking dipping.

Nitric Oxide [NO], a paramagnetic species, loses an electron rather easily, to form NO⁺, a reactive species. This reductive tendency is in contrast to the oxidative tendency of chlorine dioxide [ClO₂], another paramagnetic molecule which is a degradation product of chlorous acid. Therein lies a possible reason for the lower corrosion potential of the acidified nitrite system vs. that of acidified sodium chlorite. It is not known, at this point, what aspect(s) of the acidified nitrite system is/are the source of the antimicrobial activity which we have established for this composition, although it appears reasonable that the NO and NO⁺ components play a significant role.

Without being limited by way of theory, it is believed that the storage-stable compositions according to the present invention provide longer duration activity as a consequence of holding the pH of the composition within a set range, and holding the concentration of acid and nitrite salt within useful and effective ranges. There will be a tension between initial HONO formation and antimicrobial activity and the ability of the composition to maintain effectiveness beyond an initial period of at least 24 hours, preferably at least about 72 hours, more preferably at least about 168 hours (one week) or at least about 500 hours (about three weeks). In the acidified NO₂⁻/HONO system, i.e. NO₂⁻+H⁺< - - - >HONO, where only a fraction of the NO₂⁻ has been converted to the HONO germicidal source, when the latter has been depleted or consumed in solution, additional HONO forms from the residual NO₂⁻. The greater the degree of initial conversion, as a function of the system's pH, the lower the reservoir of NO₂⁻ and the lower the absolute amount of HONO that can subsequently form. But the greater the initial HONO, the greater the potential cidal activity that is available for the system initially. And the greater the potential for the HONO in the system to degrade, inasmuch as stability depends on HONO concentration. Conversely, the lower the initial HONO the greater the reservoir of NO₂⁻, the greater the stability (i.e. prolonged germicidal activity) but the less the cidal activity will be displaced. Thus, the use of acid activating systems which provide a reservoir of [H⁺] ions, such as a-hydroxy acids, or phosphoric acid, which are not fully ionized initially, allows for additional [H⁺] ions to combine with the NO₂⁻ in the reservoir over longer periods of time. Of course, for applications where only initial activity is needed, even mineral acids can serve as the proton source, by selecting their concentrations such that the pH of the system lies in about the 2.5 to 5.0 range.

In certain embodiments of the invention which are directed to a one-part storage-stable composition, the nitrous acid generating composition comprises from about 0.03 to about 0.70, and preferably from about 0.05 to about 0.50 percent by weight of metal nitrite, and an effective amount of an acid having a pKa of from about 2.1 to about 4.8 to maintain the pH of the composition at less than about 4.5, more preferably from about 2.5 to about 4.0. In certain aspects of the invention, the amount of acid ranges from about 0.03% to about 3.0% by weight of the final composition, but an amount of acid outside of this range may be used, depending upon the amount of nitrite salt used, as well as the desired antimicrobial activity and/or storage stability (length of time maintaining activity) desired. At metal nitrite levels higher than about 0.7%, the concentration of nitrous acid formed upon admixture of a protic acid, in the typical pH range specified, may be in excess of that required for the formation of a metastable nitrous acid teat dip solution. These higher concentrations of nitrous acid could promote the formation of nitrous oxide, and nitric oxide therefrom, through the degradation of nitrous acid at too rapid a rate, viz.
3HNO₂< - - - >H⁺+2NO+NO₃⁻+H₂O; then: 2NO+O₂(air)< - - - >2NO₂

Such solutions would not be preferred for use as teat dips or sprays, where extended lifetime (i.e., storage stability) of at least one day, are generally preferred.

Any protic acid, or acidic environment otherwise created, may be used in the present invention so long as the nitrite ion concentration limits described above are met. Suitable protic acids include such inorganic acids as phosphoric acid, and such a-hydroxy organic acids as citric, malic, lactic, tartaric, glycolic, mandelic or other structurally similar acids as described in Formula 1 hereinabove and hereinbelow, for convenience. The pKa of these organic acids may be generally from about 2.8 to about 4.2, and preferably from about 3.0 to about 4.0. Also suitable are such other acids as salicylic acid and acetic acid.

In certain preferred embodiments of the invention, an acid is used of the formula:

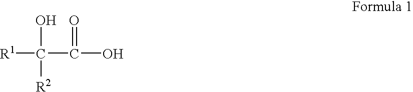

where R¹ and R² may be the same or different and may be selected from the group consisting of hydrogen, methyl, —CH₂COOH, —CH₂COO⁻, —CH₂OH, —CHOHCOOH, —C₆H₅, and —CH₂C₆H₅. The pKₐ of the organic acid is preferably from about 2.8 to about 4.8.

The amount of acid used in these teat care compositions should be sufficient to lower the pH of the composition to less than about 4.5, typically from about 2.5 to about 4, and preferably from about 2.5 to about 3.5. Single acids are generally used, but combinations may be used as well. The range of compositions of the teat dips is broad, of course, since useful acids range from the relatively weak, such as acetic acid with a pKa of 4.76, to the moderately strong, such as tartaric acid with a first pKₐ of 3.03 and phosphoric acid with a first pKₐ of 2.15. Singly-used mineral acids, which at very low levels are capable of generating disinfecting compositions, are of limited use in the inventive teat dips, since their limited H⁺ reservoir capacity for maintaining continuous optimum pH buffering predisposes to dips with lifetimes of insufficient duration.

While any metal nitrite is useful in the present composition, the alkali and alkaline earth nitrites are preferred because they are readily soluble, readily available and inexpensive. Sodium nitrite, potassium nitrite and ammonium nitrite are preferred. Sodium nitrite is particularly preferred.

A mixed acidified nitrite teat care composition will generally contain a number of other components to facilitate the benefits of the disinfecting composition. The teat dip will generally be an unthickened, slightly- or significantly-thickened colored aqueous solution, in which water represents a sufficient enough component that the normal equilibrium of the nitrite ion and nitrous acid may exist. The teat dip may be colored (as with e.g. FD&C Yellow #5 and Yellow #6) or not, and may contain other additives such as chelating agents (e.g. Na₂H₂EDTA), surfactants (e.g. alkyl aryl sulfonates such as Nacconol, and nonionic polyoxyalkylene nonylphenols such as Triton N-101), preservatives (e.g. sodium benzoate), gelling agents or thickeners (e.g. cellulose ethers or xanthan), film-forming agents (e.g. polyvinylpyrollidone, among others), and opacifying agents or opacifiers (e.g. titanium dioxide or other opacifying complexes such as those opacifying complexes formed by the reaction of cationic organic quaternary ammonium compounds (“quats”) and anionic organic sulfonates, among numerous others.

The nitrous-acid generating compound, i.e. the metal nitrite, is generally kept separate from the acid prior to use, in order to avoid premature reaction of the ingredients. Thus, in a general aspect of the present invention, compositions prior to formation of nitrous acid are found in a two part mixture. In general, once the two parts are mixed, there is an initial formation of nitrous acid followed by degradation of the nitrous acid, at a rate dependent on such factors as time, temperature, pH ands concentration of the nitrous acid. The latter will depend upon both the absolute concentration of nitrite ion and the acidity of the system, which determines the degree to which the nitrite ion is converted to nitrous acid, as demonstrated in Table 1 hereinabove. At the upper range of pH values, of about 4.5, the solutions may provide low levels of germicidal activity for several months. At somewhat lower range of pH values, of about 3.5, the high initial cidal capacity of the resulting solution may diminish with time, or even increase, as noted earlier. The molar concentration of nitrite in the teat dip solution apparently plays a role in that change. As noted in an example described hereinbelow, where the total nitrite concentration of one test solution [not a teat dip, per se] was 0.045 M/liter, the initial solution at a pH of ˜3.7 actually increased in cidal capacity. It demonstrated an E. coli kill actually greater at 20 days after mixture, than the initial kill. And in a further example, it will be noted that a barrier teat dip of the inventive composition, demonstrated an organism kill two weeks after preparation, equal to or better than the initial very high 8.5 log kill in 1 minute of contact.

The pre-mixes may also be combined by in situ application of the individual parts. They may also be applied to the various substrates associated with milking, such as teats and milking equipment, in a manner known to those skilled in this art. They may be dipped, sprayed, coated or applied in any other manner depending upon the substrate being treated. The term “substrate” as used in the instant specification is intended to cover any type of surface or carrier which could provide a locus for the accumulation of germs (bacteria, yeasts, molds, viruses, —i.e., all types of infectious agents).

Antimicrobial action may be enhanced or extended by inclusion of a variety of agents in either of the pre-mix acid or metal-nitrite compositions, or in the final mixture. These agents may include surface active materials, chelating agents, effervescent compounds and thickeners. These materials must have a minimum tendency to react with the nitrous acid system, or the acidic materials, and be compatible with the other materials in the solutions. The surface active agents, or “surfactants” may be selected from the range of available classes, but non-ionic and anionic surfactants are particularly effective. The amount of surfactant, on the final mix basis, is preferably in the range of about 0.001% to about 2%, more preferably no more than about 0.10% within this range, the level depending on the nature and effectiveness of the material in reducing the surface tension of the composition for the desired application. When the surfactant is included in the teat dip to provide supplemental antimicrobial action, such as from alkyl aryl sulfonates, the use level may be several orders of magnitude higher.

Preservatives may also be used in either or both of the pre-mix compositions, to stabilize the solutions. On the basis of the total composition the amount of preservative, from both pre-mix compositions if so present, may generally be from about 0.01 to about 0.08, typically from about 0.01 to about 0.06, and preferably from about 0.02 to about 0.04 percent by weight of the total composition. When the preservative, however, is included in the teat care composition to provide supplemental antimicrobial action, such as from sodium benzoate which converts to germicidal benzoic acid in an acid environment, the use level may be several orders of magnitude higher.

When these compositions are used as so-called “barrier” teat dips, they are typically applied as thickened solutions to facilitate adherence to the skin, and facilitate a greater laydown of germicide. Any thickener or gelling agent which is non-toxic and non-reactive with the nitrous acid system may be used. Many carbohydrate polymers are possible candidates, although some such as the cellulose-based thickeners are less preferred because of their tendency to oxidatively cleave at the β-D-glucose linkage. A preferred thickener is xanthan gum, which is minimally reactive in both the individual pre-mix composition and the final acidified nitrite mix. Other appropriate thickeners include those based on poly(oxyalkylenes) and poly(acrylamides) the latter including the sulfonic acid derivatives thereof, and mineral thickeners such as the silica-based and clay gelling agents. Materials such as the poly(acrylamides), and co-polymers thereof, can function as well as to create a skin-like covering on the teat, as the teat dip dries. Polymers such as poly(vinyl alcohol), which are non-thickening per se will similarly form pseudo-skins on the teat as the result of drying thereon.

The amount of thickener or gelling agent which may be used in the thickened, gel composition will vary, depending upon the thickening properties of the gelling agent, the intended application, the level and nature of the acid, the level of the metal nitrite, and other additives employed. Generally, the amount may be from about 0.5 to about 30, typically from about 1 to about 15, and preferably about 1 to about 12 percent by weight of the total composition. Different thickeners may be used in each part of the pre-mix composition, and these levels refer to the combined levels of gelling agent in the total composition. Film-forming agents may be used at about the same concentrations as thickeners or gelling agents in the present compositions.

The amount of metal nitrite in the nitrous-acid generating pre-mix is adjusted so that when the solution (including thickened liquid) is mixed with the acidic component, the specified percentage of metal nitrite will be present in the resulting composition. For example, when two thickened pre-mixes are designed to be mixed in equal parts, which is preferred, the amount of metal nitrite in one part may be generally from about 0.02% to about 2%, typically from about 0.04% to about 1%, and preferably from about 0.06% to 0.6% by weight of that part. Similarly, the amount of acid in the counterpart pre-mix should be sufficient such that when that pre-mix is combined with the metal nitrite pre-mix, the pH of the resulting composition will be less than about 4.5, typically from about 2.5 to about 4, and preferably from about 2.5 to about 3.5. The wide diversity of possible acid sources is such that no particular weight specification for amounts of acid is feasible except on a case-by-case basis, although the acid is used in the present invention in effective amounts.

The two teat-dip pre-mix liquids may be combined just prior to application, or up to at least several weeks before use, or they may be simultaneously mixed and applied in situ. The teat care compositions may be dipped, sprayed, or may be coated onto teats by techniques known to those skilled in dairy practices, or applied in any other manner depending upon the needs of the dairy practitioner or farmer.

In certain preferred embodiments of the present invention a disinfectant composition comprises a single-phase liquid or gel comprising nitrous acid and an a-hydroxy acid, wherein the pH of the composition either remains relatively constant at an initial value of around 3.7 or lower, or decreases from said initial value of around 3.75 or lower at the time of formulation to a value as low as around 2.5 over a period of at least about two days, preferably about two days to five days; the molar percentage of nitrite ion in the composition in the form of nitrous acid is greater than about 35% but less than about 95% of the total nitrite ions present in the composition; and the composition exhibits cidal activity against microorganisms for a period of at least three weeks (preferably at least about two months or at least three months) after formulation.

The present invention is illustrated by the following Examples. Examples 1 through 5, and Example 9 illustrate the basic germicidal capabilities of the acidified nitrite systems, and Examples 6,7 and 8 demonstrate the functionality of acidified nitrite teat care systems. All parts and percentages in the Examples, as well as the specifications and claims, are by weight, unless otherwise specified. The following examples, which are non-limiting, further describe preferred embodiments within the scope of the present invention. Many variations of these Examples are possible without departing from the spirit of the invention.

### EXAMPLE 1

This example illustrates the ability of six acidified nitrite solutions to destroy high levels of the Gram-positive organism Staphylococcus aureus (ATCC 29213), and to a degree consistent with the relative percentage of nitrous acid with respect to total nitrite in the solution. The mixed nitrite/acid solutions, their resulting pH values, and the relative percentages of nitrous acid in the solutions were as shown below. To prepare these solutions, equal parts of a 0.625% NaNO₂ solution and increasing concentrations of malic acid solution were combined as follows:

| | | Malic | | Total Nitrite |
|---|---|---|---|---|
| Sol'n No. | NaNO₂ Premix | Acid Premix | Mix pH | as Nitrous Acid |
| 1 | 0.625% | 2.25% | 2.94 | 70% |
| 2 | 0.625% | 1.225% | 3.12 | 60% |
| 3 | 0.625% | 0.812% | 3.35 | 47% |
| 4 | 0.625% | 0.419% | 3.54 | 37% |
| 5 | 0.625% | 0.263% | 3.75 | 28% |
| 6 | 0.625% | 0.156% | 3.90 | 21% |

Procedure: A heavy suspension of the S. aureus was prepared in saline, and 1 part of the suspension was separately combined with 10 parts of each of the above solutions, which had been prepared five minutes before the testing. After five minutes of contact, the mixtures were added to nine volumes of Dey/Engley broth to neutralize the activity and acidity. A 10-fold dilution in saline was made of this mixture. 2 mls of the sample diluted in D/E broth were added to each of five petri plates. 1 ml of the sample diluted in D/E broth was added to each of two petri plates, and 1 ml of the 1/10 dilution of the sample diluted in D/E broth was added to each of two petri plates. Approximately 10 mls of semisolid Trypticase Soy Agar were added to each petri plate, swirled and allowed to harden. The plates were incubated at 350-37° C. for 48 hours, and the resulting colonies were enumerated.

The number of microorganisms in the original suspension was determined by making ten-fold dilutions from 10⁻¹ to 10⁻⁸. Then 1.0 ml portions of the 10⁻⁷ suspension were added to each of two sterile petri plates. 1.0 ml of the 10⁻⁸ suspension was added to each of two sterile petri plates, and 0.1 ml of the 10⁻⁸ suspension was added to each of two sterile petri plates. Approximately 10 mls of semisolid agar were added to each petri plate, swirled and allowed to harden. The plates were incubated at 35°-37° C. for 48 hours, and the resulting colonies were enumerated.

Results:
S. aureus Cidal Data*

| Sol'n No. | Recovered cfu | Log Recovery | Log Kill |
|---|---|---|---|
| 1 | 5.4 × 10¹ | 1.7 | 9.1 |
| 2 | 7.0 × 10³ | 3.8 | 7.0 |
| 3 | 4.5 × 10³ | 3.6 | 7.2 |
| 4 | 5.6 × 10⁴ | 4.7 | 6.1 |
| 5 | 6.6 × 10⁵ | 5.8 | 5.0 |
| 6 | >1 × 10⁶ | >6.0 | <4.8 |

*Inoculum suspension contained 10.8 logs of organisms.

It is obvious that a)—there was significant destruction of the high inoculum of S. aureus in the 5-minute contact period, and b)—the degree of destruction closely parallels the degree of conversion of the nitrite ion to nitrous acid. A 9.1 log kill (>1 billion-fold) was achieved with a solution in which 70% of the nitrite existed in its acidified form of nitrous acid, whereas only 5.0 logs (100,000-fold) were destroyed by the solution with nitrous acid representing 28% of the total nitrite. Even less was destroyed in the 21% nitrous acid (relative) solution.

### EXAMPLE 2

This example illustrates the ability of six acidified nitrite solutions to destroy high levels of the Gram-negative organism Escherichia coli (ATCC 25922). The procedure described in Example 1 was applied in this study as well, using aliquots of the same solutions described in the Table.

The results were as follows:

Results:
E. coli Cidal Data*

| Sol'n No. | Recovered cfu | Log Recovery | Log Kill |
|---|---|---|---|
| 1 | 2.7 × 10² | 2.4 | 7.7 |
| 2 | 6.6 × 10⁴ | 4.8 | 5.3 |
| 3 | 9.0 × 10⁰ | 1.0 | 9.1 |
| 4 | 1.4 × 10¹ | 1.1 | 9.0 |
| 5 | 9.9 × 10² | 3.0 | 7.1 |
| 6 | 3.1 × 10³ | 3.5 | 6.6 |

*Inoculum suspension contained 10.1 logs of organisms.

In the case of this Gram-negative organism, the destruction of the inoculum was high in all solutions, apparently independent of pH and thus the relative amount of total nitrite existing as nitrous acid in this series of solutions. It is not known, at this point, whether this difference with respect to the observations in Example 1 is characteristic of the kill mechanism of acidified nitrite solutions with respect to Gram-positive and Gram-negative organisms, or whether it relates to these particular organisms

### EXAMPLE 3

This example illustrates the ability of six acidified nitrite solutions to destroy high levels of the Gram-negative organism Escherichia coli (ATCC 25922), following 20 days of storage of the mixed solutions at ambient temperatures prior to the testing. The procedure described in Example 1 was applied in this study as well, using aliquots of the same solutions that were evaluated in Examples 1 and 2. The results were as follows:

Results:

The data are presented in the following Table, in which the kills measured on the 20-day old solutions are compared with data obtained on the T=0 mixtures (in brackets).

E. coli Cidal Data on 20-day aged mixtures*

| Sol'n No. | Recovered cfu | Log Recovery | Log Kill** |
|---|---|---|---|
| 1 | 6.0 × 10¹ | 1.8 | 9.2 [7.7] |
| 2 | 1.5 × 10² | 2.2 | 8.8 [5.3] |
| 3 | 6.0 × 10⁰ | 0.8 | 10.2 [9.1] |
| 4 | >1 × 10⁵ | >5.0 | <6.0 [9.0] |
| 5 | 3.2 × 10⁴ | 3.5 | 7.5 [7.1] |
| 6 | >1 × 10⁶ | >6.0 | <5.0 [6.6] |

*Inoculum suspension contained 11.0 logs of organisms.
**Bracketed data are log kills at T = 0 with the same solutions

About three weeks after preparation, the mixed solutions have retained a significant cidal capacity, as compared with their abilities at T=0. In fact the pH's of these aged solutions, as cf. their original values, sheds some light on the greater cidal capacity of the first few solutions tested, viz.

| pH at T = 0 | pH at T = 30 days |
|---|---|
| 2.94 | 2.30 |
| 3.12 | 2.50 |
| 3.35 | 3.25 |
| 3.54 | 3.15 |
| 3.75 | 3.92 |
| 3.90 | 4.35 |

The highest activity, in both fresh and aged solution, appears to occur in the solutions where the pH levels dropped, leading to higher levels of nitrous acid. In these solutions, the nitrous acid and nitrite exist in a ratio of ca. 1:1 and higher. This leads to the speculation that the stability (as well as the activity) of these solutions is related to the presence of a complex ion, such as [HN₂O₄]⁻, analogous to the [Cl₂O₄]⁻ found to exist in ClO₂/ClO₂⁻ systems, where the complex [Cl₂O₄]⁻ is conjectured to be an active cidal species, of a higher oxidation potential than ClO₂ alone.

### EXAMPLE 4

This example illustrates the ability of six acidified nitrite solutions to destroy high levels of the yeast Candida albicans (ATCC 10231), and to a degree consistent with the relative percentage of nitrous acid with respect to total nitrite in the solution. The mixed nitrite/acid solutions, their resulting pH values, and the relative percentages of nitrous acid in the solutions were similar to those shown in Example 1.

Procedure: A heavy suspension of the C. albicans was prepared in saline, and 1 part of the suspension was separately combined with 10 parts of each of the above solutions, which had been prepared five minutes before the testing. After five minutes of contact, the mixtures were added to nine volumes of Dey/Engley broth to neutralize the activity and acidity. A 10-fold dilution in saline was made of this mixture. 2 mls of the sample diluted in D/E broth were added to each of five petri plates. 1 ml of the sample diluted in D/E broth was added to each of two petri plates, and 1 ml of the 1/10 dilution of the sample diluted in D/E broth was added to each of two petri plates. Approximately 10 mls of semisolid Sabouraud Dextrose Agar were added to each petri plate, swirled and allowed to harden. The plates were incubated at 20°-25° C. for 72 hours, and the resulting colonies were enumerated.

The number of microorganisms in the original suspension was determined by making ten-fold dilutions from 10⁻¹ to 10⁻⁸. Then 1.0 ml portions of the 10⁻⁷ suspension were added to each of two sterile petri plates. 1.0 ml of the 10⁻⁸ suspension was added to each of two sterile petri plates, and 0.1 ml of the 10⁻⁸ suspension was added to each of two sterile petri plates. Approximately 10 mls of semisolid agar were added to each petri plate, swirled and allowed to harden. The plates were incubated at 20°-25° C. for 72 hours, and the resulting colonies were enumerated.

C. albicans Cidal Data*

| Sol'n No. | Recovered cfu | Log Recovery | Log Kill | % HONO** |
|---|---|---|---|---|
| 1 | 0 | 0 | >7.86 | 70 |
| 2 | 4 | 0.6 | 7.26 | 60 |
| 3 | 2.4 × 10¹ | 1.38 | 6.48 | 47 |
| 4 | 2.1 × 10⁴ | 4.32 | 3.54 | 37 |
| 5 | >1 × 10⁶ | >6 | <˜1 | 28 |
| 6 | >1 × 10⁶ | >6 | <˜1 | 21 |

*Inoculum suspension contained 7.86 logs of organisms.
**% of total nitrite ion present as nitrous acid

The destruction of the C. albicans yeast is quite significant, particularly for the solutions below about pH 3.5, where the nitrous acid is present in a ratio of about 1:1 with respect to ionic nitrite (i.e., above about 50% of total nitrite as HONO). Thereafter the fall off in kill is rather dramatic, at higher pHs. For this organism, as for the S. aureus of Example 1, this suggests that a 1:1 adduct of nitrous acid and nitrite may be providing particularly effective cidal capacity in this system.

### EXAMPLE 5

This example illustrates the ability of six acidified nitrite solutions to destroy high levels of the mold Aspergillus niger (ATCC 6275). The mixed nitrite/ acid solutions, their resulting pH values, and the relative percentages of nitrous acid in the solutions were similar to those shown in Example 1, and the procedure followed paralleled that provided in Example 4.

A. niger Cidal Data*

| Sol'n No. | Recovered cfu | Log Recovery | Log Kill |
|---|---|---|---|
| 1 | 18 | 1.26 | 7.14 |
| 2 | 83 | 1.92 | 6.48 |
| 3 | 30 | 1.48 | 6.92 |
| 4 | 37 | 1.57 | 6.83 |
| 5 | 0 | 0 | >8.40 |
| 6 | 0 | 0 | >8.40 |

*Inoculum suspension contained 8.40 logs of organisms.

### EXAMPLE 6

This example illustrates the high level and duration of efficacy of an acidified nitrite teat dip composition against the Environmental organism E. coli (ATCC 25922). An in vitro microbiological evaluation was run on the composition at three times; when freshly mixed as well as 1 day and 2 days after preparation. The two components of the teat dip were as follows:

| | |
|---|---|
| Nitrite Base: | |
| Sodium nitrite | 0.625% |
| Sodium dodecylbenzene sulfonate | 0.20% |
| FD&C Yellow #5 | 0.20% |
| Water | q.s. |
| Acid Activator: | |
| Lactic acid (88%)* | 3.23% |
| Glycerin | 10.0% |
| Natrosol 250MR | 0.50% |
| Sodium benzoate | 0.04% |
| Benzalkonium chloride (17%) | 1.26% |
| Water | q.s. |

*HCl was added so that a 1:1 mix of both parts had a pH of 2.95.

Procedure: The initial inoculum at each test period was >10⁸, as will be seen in the test data. The microorganism was plated on Trypticase Soy Agar and incubated at 35°-37° C. for 24 hours. A heavy suspension was prepared in sterile saline. Equal quantities (by weight) of the teat dip components were mixed together, and allowed to stand for about 10 minutes. Then nine volumes of this sample was challenged with one volume of the organism suspension for 15 seconds. Then 2.0 ml of the mixture were added to 18 ml of D/E broth. A further 1/10 dilution of the D/E broth in saline was prepared. Five 2.0 ml samples of the D/E broth were added to petri plates. Duplicate 1.0 ml samples were added to petri plates, and duplicate 1.0 ml samples of the 1/10 dilution were added to petri plates. Approximately 10 ml of liquid Trypticase Soy Agar were added to each petri plate and allowed to solidify. Plates were incubated at 35°-37° C. for 24-48 hours, and colony forming units were counted. Thereafter the mixed sample was incubated in a foil-covered sterile container at room temperature, until use. After the first sample (Day 0) sample was tested, samples were removed for testing 1 and 2 days after mixing (Day 1 and 2, resp.) and were tested as above. At each test point a control study was run, in which a sample of saline was challenged, instead of the test sample.

Results:

| | | | |
|---|---|---|---|
| | Challenge Inoculum | Organisms | |
| | (Log #cfu/ml | Recovered (Log | Log |
| Test Sample | Product) | #cfu/ml Product | Reduction |

Day 0

| | | | |
|---|---|---|---|
| Teat Dip | 7.8 × 10⁸ (8.89) | 0 | >8 89 |
| Control (Saline) | 7.8 × 10⁸ (8.89) | 3.8 × 10⁸ (8.89) | — |

Day 1

| | | | |
|---|---|---|---|
| Teat Dip | 5.3 × 10⁸ (8.72) | 1.7 × 10¹ (1.23) | 7.47 |
| Control (Saline) | 5.3 × 10⁸ (8.72) | 5.1 × 10⁸ (8.70) | — |

Day 2

| | | | |
|---|---|---|---|
| Teat Dip | 3.4 × 10⁸ (8.53) | 0 | >8.56 |
| Control (Saline) | 3.4 × 10⁸ (8.53) | 3.6 × 10⁸ (8.56) | — |

These results clearly demonstrate that the acidified nitrite teat dip was capable of destroying upwards of 100 million E. coli organisms within 15 seconds of contact, up through two days following mixture. The 17 remaining organisms, of the 530 million challenge at Day 1, are considered artifactual, in as much as the 2-day aged sample destroyed all of the challenge. It is evident from these data that acidified nitrite antimicrobials can exert continued cidal activity against mastitis-causing microorganisms long after their initial preparation.

### EXAMPLE 7

This example illustrates the prolonged high-level efficacy of a thickened version of the above acidified nitrite teat dip composition against the Environmental organism E. coli (ATCC 25922). This type of teat dip is generally termed a “barrier” dip, because it deposits a protective film on the teat during and after drying, so as to protect the teat during the intermilking period. The composition provided in Example 6 was modified by the addition of two components to the nitrite base, specifically 0.50% xanthan gum and 2.24% of Fixomer A-30, a 70/30 copolymer of methacrylic acid and poly(acrylamidomethyl propane sulfonic acid). In this study, in vitro microbiological evaluations were run on the composition at five times; when freshly mixed as well as 1, 2, 6 and 14 days after preparation. The procedure was the same as in Example 6, except that a 1-minute contact was used for the studies, based on the extended contact of a barrier dip, which is applied post-milking, and intended to last on the teat for up to ˜12 hours until the next milking.

Results:

| | | | |
|---|---|---|---|
| | Challenge | | |
| | Inoculum (Log | Organisms Recovered | Log |
| Test Sample | #cfu/ml Product) | (Log #cfu/ml Product) | Reduction |

Day 0

| | | | |
|---|---|---|---|
| Teat Dip | 2.2 × 10⁸ (8.34) | 0 | >8.40 |
| Control (Saline) | 2.2 × 10⁸ (8.34) | 2.5 × 10⁸ (8.40) | — |

Day 1

| | | | |
|---|---|---|---|
| Teat Dip | 4.0 × 10⁸ (8.60) | 1.7 × 10¹ (1.23) | 7.20 |
| Control (Saline) | 4.0 × 10⁸ (8.60) | 2.7 × 10⁸ (8.43) | — |

Day 2

| | | | |
|---|---|---|---|
| Teat Dip | 3.4 × 10⁸ (8.53) | 0 | >8.38 |
| Control (Saline) | 3.4 × 10⁸ (8.53) | 2.4 × 10⁸ (8.38) | — |

Day 6

| | | | |
|---|---|---|---|
| Teat Dip | 3.2 × 10⁸ (8.51) | 0 | >8.61 |
| Control (Saline) | 3.2 × 10⁸ (8.51) | 4.1 × 10⁸ (8.61) | — |

Day 14

| | | | |
|---|---|---|---|
| Teat Dip | 7.8 × 10⁸ (8.89) | 0 | >8.58 |
| Control (Saline) | 7.8 × 10⁸ (8.89) | 3.8 × 10⁸ (8.58) | — |

These results clearly demonstrate that the acidified nitrite barrier teat dip was capable of destroying 220-780 million E. coli organisms within 60 seconds of contact, up through two weeks following mixture. As demonstrated in Example 6, and further evident from these data, acidified nitrite teat dips can exert continued and very high cidal activity against mastitis-causing microorganisms long after the teat dip's initial preparation.

### EXAMPLE 8

This example demonstrates the antimicrobial activity of the teat dip composition described in Example 6 against the Contagious microorganism Staph. aureus [ATCC 29213], using a teat model system. In this test, four wooden birch dowels are used, for both test and control samples, to simulate animal teats. The microorganism suspension was prepared by inoculating 50 ml of Trypticase Soy Broth (TSB), and incubating at 35°-37° C. for 18-24 hours. The wooden dowels, (ca. 0.6 inch diameter×2 inch length) were fitted with screw hooks attached at one end at approximately a 45° angle, to facilitate dripping of excess teat dip. The dowels were marked at a point one inch from the end opposite the hook, and then covered with the finger of a latex glove. Each dowel was then suspended from a wire.

At the beginning of each experiment, each dowel was sprayed with 70% isopropyl alcohol and allowed to dry for ca. 10 minutes. Following this, each dowel was dipped into the microorganism suspension, up to the one inch mark. The dowels were again allowed to dry for approximately 10 minutes. 15 ml of each of the teat dip components were mixed for approximately 15 seconds, and allowed to stand for about 5 minutes. The dowels were then dipped into the teat dip, past the one-inch mark, to ensure that all of the dried microorganism had been covered. This was replicated on the remaining four simulated teats. After a one-minute contact, the dowels were submerged past the one-inch mark in 18 ml of D/E broth. A 1/10 dilution of the D/E broth was prepared in saline.

Duplicate 1.0 ml samples of the D/E broth were placed into each of two petri plates. Duplicate 1.0 ml samples of the 1/10 dilution of the D/E broth were placed into each of the two petri plates, and duplicate 0.1 ml samples of the 1/10 dilution of the D/E broth were placed into each of two petri plates. Approximately 10 ml of liquid Trypticase Soy Agar was added to each petri plate and allowed to solidify. Plates were incubated at 35°-37° C., for 24-48 hours, and colony forming units were counted. Log reductions of the test dip challenges were calculated with respect to the organisms present in the Control saline. Although the initial S. aureus challenge contained 7.2×10⁸ organisms per ml, the saline itself physically removes several logs worth of organisms from the teat model, so the reference quantity is considerably smaller (as can be seen from the following data tabulation).

| | | | | | Geometric | Reduction vs. |
|---|---|---|---|---|---|---|
| Sample | Teat 1 | Teat 2 | Teat 3 | Teat 4 | Average | Control |
| Test | 0 | 0 | 0 | 0 | 0 | 5.4 logs |
| Control | 2.8 × 10⁵ | 2.6 × 10⁵ | 2.2 × 10⁵ | 2.5 × 10⁵ | 2.8 × 10⁵ (5.4 logs) | — |

These data confirm that the teat dip comprised of an acidified nitrite solution, is an effective cidal agent against a microorganism strongly associated with Contagious mastitis in dairy cows.

### EXAMPLE 9

This example illustrates the ability of one of the six nitrous acid solutions tested in Examples 1 through 5, specifically Solution No. 2, to be as microbiocidally effective after over two (2) years of storage at ambient temperatures, as it was in both Example 2 (the day of preparation) and Example 3 (after 20 days of ambient storage). In Example 2 the nitrous acid, formulated with equal parts of 0.625% NaNO₂ and 1.225% Malic Acid, was shown to destroy 5.3 logs of the Gram-negative organism Esciherichia coli (ATCC 25922) after 5 minutes of contact. In Example 3, after 20 days of storage, the aged solution destroyed 8.8 logs of that organism.

After over 26 months of ambient storage (specifically 735 days), an aliquot of that nitrous acid solution was tested for its 5-minute kill, with the following results:

Test Organism: E. coli ATCC 25922
Initial Suspension: 1.4 × 10⁹

| | Challenge | Recovered | |
|---|---|---|---|
| | Inoculum (Log | (Log | Log |
| Test Sample | cfu/ml) | cfu/ml) | Reduction |
| Sample mixed on Sep. 28, 2001 | 1.4 × 10⁸ | 0 | >8.15 |
| and stored at room temp. | (8.15 logs) | | |
| Control (Saline) | 1.4 × 10⁸ | 1.1 × 10⁸ | — |

The procedure for E. coli was the same as described in the earlier Examples as follows:

The microorganism was plated on Trypticase Soy Agar and incubated at 35-37° C., for 18-24 hours. A heavy suspension was prepared in sterile saline. The challenge sample, which had been mixed on Sep. 28, 2001, had been stored in a capped glass test tube at room temperature until testing. Nine volumes of the sample (1.8 ml) were challenged with one volume (0.2 ml) of the organism for 5 minutes. Following this 2.0 ml of this mixture was added to 18 ml of D/E broth. A further 1/10 dilution of the D/E broth in saline was prepared. Five 2.0 ml samples of the D/E broth were added to petri plates. Duplicate 1.0 ml samples were added to petri plates, and duplicate 1.0 ml samples of the 1/10 dilution were added to petri plates. Approximately 10 ml of liquid Trypticase Soy Agar was added to each petri plate and allowed to solidify. Plates were incubated at 35-37° C., for 24 hours, and colony forming units were counted. A control study was run, in which a sample of saline was challenged, instead of the test sample.

This Example clearly demonstrates that this nitrous acid solution, at a pH below about 3.4 (as deducted from the aging data in Example 3 and the pH information provided in Example 1), is capable of providing a high level of antimicrobial activity, for at least several years after its formation, when stored under ambient conditions.

It is clear that the present invention is well adapted to carry out the objects, and achieve the ends and advantages mentioned at the outset. While currently preferred embodiments of the invention have been described for purposes of this disclosure, numerous modifications may be made which will readily suggest themselves to those skilled in the art, and which are encompassed within the spirit of he invention disclosed, and as defined in the appended claims.

References Cited
US Patent Documents

| Number | Issue Date | Inventor |
|---|---|---|
| 4084747 | April, 1978 | Alliger |
| 4330531 | May, 1982 | Alliger |
| 4891216 | January, 1990 | Kross et al. |
| 4956184 | June, 1988 | Kross |
| 4986990 | January, 1991 | Davidson etal. |
| 5100652 | May, 1992 | Kross et al. |
| 5185161 | February, 1993 | Davidson et al. |
| 5384134 | January, 1995 | Kross, et al. |
| 5389390 | February 1995 | Kross |
| 5597561 | January, 1997 | Kross |
| 5628959 | May, 1997 | Kross |
| 5651977 | July, 1997 | Kross |
| 5772985 | June, 1998 | Kemp, et al. |
| 5820822 | October, 1998 | Kross |
| RE36,064 | January, 1999 | Davidson et al. |
| 6063425 | May, 2000 | Kross |
| 6096350 | August, 2000 | Kemp, et al. |
| 6099881 | August, 2000 | Hanson |
| 6123966 | September, 2000 | Kross |

Other Patent Documents

| | |
|---|---|
| PCT Application WO95/22335 Feb. 17, 1995 | Nigel Benjamin et al. |
| (Acidified Nitrite as an Antimicrobial Agent) | |
| PCT Application WO 02/17881 Aug. 30, 2001 | Tucker et al. |
| (Transdermal Pharmaceutical Delivery System) | |
| US Patent Appl. Pub. US 2002/0136750 A1 | Benjamin et al. |
| Sep. 26, 2002 (Acidified Nitrite as an | |
| Antimicrobial Agent) | |
| US Patent Appl. Pub. US 2002/0155174 A1 | Benjamin et al. |
| Oct. 24, 2002 (Acidified Nitrite as an | |
| Antimicrobial Agent) | |

### OTHER REFERENCES

Friedman, H. L. “On tile Ultraviolet Absorption Spectra of Uninegative Ions”, J. Chem. Physics, (1953), Vol. 21, No. 1, p. 319 et seq.
Masschelein, W J; (1979) Chlorine Dioxide; Chemistry and Environmental Impact of Oxychlorine Compounds. Ann Arbor Science, Mich.

1. A process for disinfecting mammalian teat skin comprising contacting said substrate with an aqueous teat dip composition consisting essentially of water and an effective amount of a protic acid, or a material inducing an acidic environment therein, and an effective amount of a water soluble metal nitrite to produce nitrous acid from said acid and said metal nitrite, said composition containing an amount of nitrous acid which is no more than about 95% by weight of the total amount of nitrite ion and nitrite as nitrous acid in said composition.

2. The process according to claim 1 wherein the metal nitrite is present at a level of from about 0.03% to about 0.7% by weight based on the total weight of the composition.

3. The process according to claim 1 wherein the metal nitrite is sodium nitrite.

4. The process according to claim 1 wherein the protic acid is an organic acid.

5. The process of claim 4 wherein the organic acid has a pKₐ value in the range of about 2.8 to about 4.8.

6. The process of claim 4 wherein the organic acid has a pKₐ value in the range of about 2.8 to about 4.2.

7. The process of claim 4 wherein the organic acid has a pKₐ value in the range of about 3.0 to about 4.0.

8. The process according to claim 4 wherein said acid is an α-hydroxy acid of the general formula: 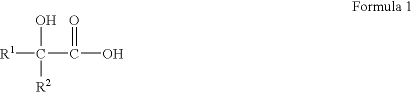
wherein R¹ and R² may be the same or different and may be selected from the group consisting of hydrogen, methyl, —CH₂ COOH, —CH₂ COO⁻, —CH₂ OH, —CHOHCOOH, —C₆H₅ , and —CH₂C₆H₅.

9. The process of claim 5 wherein the organic acid ranges from about 0.03% to about 3% by weight of the total composition.

10. The process according to claim 1 wherein said aqueous composition has a pH of less than about 4.5.

11. The process according to claim 1 wherein said aqueous composition has a pH ranging from about 2.5 to about 4.0.

12. The process according to claim 1 wherein said aqueous composition has a pH ranging from about 2.5 to about 3.5.

13. The process according to claim 1 wherein the protic acid is an inorganic acid.

14. The process of claim 13 wherein the inorganic acid is selected from the group consisting of nitric acid, hydrochloric acid, sulfuric acid, sodium hydrogen sulfate and phosphoric acid.

15. The process of claim 1 wherein the teat dip composition is a thickened composition comprising a gelling or film-forming agent.

16. The process according to claim 15 wherein said thickened composition remains on the teat upon drying of the composition thereon.

17. The process according to claim 15 wherein said gelling or film-forming agent comprises about 0.5 percent to about 30 per cent by weight of said composition, typically from about 1 percent to about 15 percent, and preferably at about 1 percent to about 12 percent.

18. The process of claim 1 wherein the aqueous teat dip composition retains significant microbiocidal activity for a period of at least about 24 hours after preparation.

19. The process of claim 1 wherein the aqueous teat dip composition retains significant microbiocidal activity for a period of at least about three weeks after preparation.

20. The process of claim 1 wherein the aqueous teat dip composition retains significant microbiocidal activity for a period of at least about two weeks after preparation.

21. The process according to claim 1 wherein the mammalian skin is that of a cow.

22. A long-acting antimicrobial composition comprising a single-phase liquid or gel comprising an antimicrobially effective amount of nitrous acid and a protic acid wherein said composition comprises about 0.03% to about 0.70% by weight of a metal nitrite and said protic acid is included in said composition in an amount effective to produce a pH of less than about 4.5 in said composition and wherein said antimicrobial activity is maintained in said composition for a period of at least about 48 hours.

23. The composition according to claim 22 wherein said acid is an α-hydroxy acid having a pKa value ranging from about 2.1 to about 4.8, said pH of said composition ranging from about 2.5 to about 4.0.

24. The composition according to claim 22 wherein said protic acid has the chemical structure: 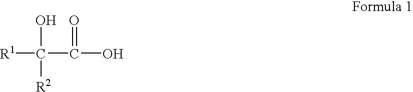
wherein R¹ and R² may be the same or different and may be selected from the group consisting of hydrogen, methyl, —CH₂COOH, —CH₂COO⁻, —CH₂OH, —CHOHCOOH, —C₆H₅, and —CH₂C₆H₅.

25. A composition according to claim 22, wherein the nitrous acid is generated by a sodium or potassium nitrite.

26. A composition according to claim 22, wherein the composition is a liquid teat dip.

27. A composition according to claim 22, wherein the composition is a gel.

28. A composition comprising a single-phase liquid or gel comprising nitrous acid and an α-hydroxy acid, wherein:
(a) the pH of the composition either remains relatively constant at an initial value of around 3.7 or lower, or decreases from said initial value of around 3.75 or lower at the time of formulation to a value as low as around 2.5 over a period of at least about two days, preferably about two days to five days;
(b) the molar percentage of nitrite ion in the composition in the form of nitrous acid is greater than about 35% but less than about 95% of the total nitrite ions present in the composition; and
(c) the composition exhibits cidal activity against microorganisms for a period of at least three weeks after formulation.

29. A composition of claim 28, wherein the composition comprises a compound comprising an amount of phosphoric acid with a pKa of about 2.15 that is sufficient to lower the pH of the composition to less than about 3.75.

30. A composition according to claim 28, wherein the a-hydroxy acid is a compound of the formula (I): 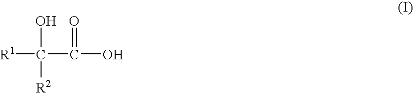
wherein R¹ and R² may be the same or different and may be selected from the group consisting of hydrogen, methyl, —CH₂COOH, —CH₂COO⁻, —CH₂OH, —CHOHCOOH, —C₆H₅, and —CH₂C₆H₅.

31. A composition according to claim 28, wherein the composition further comprises one or more of the following components: a surface active agent, a chelating agent, an effervescent compound, a preservative, a coloring agent, an opacifier and a thickener.

32. A composition according to claim 28, wherein the cidal activity of the composition over a period of about twenty-four months or more after formulation is comparable to the activity that it demonstrated initially.

33. A composition according to claim 28, wherein the cidal activity of the composition over a period of about five minutes or more after formulation is equivalent to the activity necessary to achieve an approximately eight log decrease in a sample of E. coli.

34. A composition according to claim 28, wherein the composition is used in conjunction with an application medium.

35. A composition according to claim 28, wherein the nitrous acid is generated by a metal nitrite.

36. A composition according to claim 28, wherein the composition may be used as a liquid teat dip.

37. A composition according to claim 28, wherein the composition is a gel.

38. A method comprising disinfecting a substrate by application thereto of a composition according to claim 28.

39. A method comprising disinfecting mammalian tissue by application to said tissue of a composition according to claim 28.

40. The method according to claim 38 wherein said substrate is a metal surface.

41. A method according to claim 38 wherein said composition comprises an amount of nitrite in the form of nitrous acid that is no more than about 85% by weight of the total nitrite ions in the composition.

42. The method according to claim 38, wherein the composition is a disinfecting gel comprising a thickener.

43. The method according to claim 38, wherein the composition is an oral rinse.

44. The method according to claim 38 wherein said application of said composition occurs over a period of at least about several months.

45. The method according to claim 38 wherein the substrate is mammalian tissue.

46. The method according to claim 38 wherein the composition is sprayed onto the substrate.

47-51. (canceled)
